SEQUENCE LISTING (9 sequences)

SEQ ID NO: 1 - DNA, 1017 bp, Homo sapiens
atgagtctaagtgcatttactctcttcctggcattgattggtggtaccagtggccagtactatgattatgattttcccctatcaatttatgggcaatcatcaccaaactgtgcaccagaatgtaactgccctgaaagctacccaagtgccatgtactgtgatgagctgaaattgaaaagtgtaccaatggtgcctcctggaatcaagtatctttaccttaggaataaccagattgaccatattgatgaaaaggcctttgagaatgtaactgatctgcagtggctcattctagatcacaaccttctagaaaactccaagataaaagggagagttttctctaaattgaaacaactgaagaagctgcatataaaccacaacaacctgacagagtctgtgggcccacttcccaaatctctggaggatctgcagcttactcataacaagatcacaaagctgggctcttttgaaggattggtaaacctgaccttcatccatctccagcacaatcggctgaaagaggatgctgtttcagctgcttttaaaggtcttaaatcactcgaataccttgacttgagcttcaatcagatagccagactgccttctggtctccctgtctctcttctaactctctacttagacaacaataagatcagcaacatccctgatgagtatttcaagcgttttaatgcattgcagtatctgcgtttatctcacaacgaactggctgatagtggaatacctggaaattctttcaatgtgtcatccctggttgagctggatctgtcctataacaagcttaaaaacataccaactgtcaatgaaaaccttgaaaactattacctggaggtcaatcaacttgagaagtttgacataaagagcttctgcaagatcctggggccattatcctactccaagatcaagcatttgcgtttggatggcaatcgcatctcagaaaccagtcttccaccggatatgtatgaatgtctacgtgttgctaacgaagtcactcttaattag

SEQ ID NO: 2 - protein, 338 aa, Homo sapiens
MSLSAFTLFLALIGGTSGQYYDYDFPLSIYGQSSPNCAPECNCPESYPSAMYCDELKLKSVPMVPPGIKYLYLRNNQIDHIDEKAFENVTDLQWLILDHNLLENSKIKGRVFSKLKQLKKLHINHNNLTESVGPLPKSLEDLQLTHNKITKLGSFEGLVNLTFIHLQHNRLKEDAVSAAFKGLKSLEYLDLSFNQIARLPSGLPVSLLTLYLDNNKISNIPDEYFKRFNALQYLRLSHNELADSGIPGNSFNVSSLVELDLSYNKLKNIPTVNENLENYYLEVNQLEKFDIKSFCKILGPLSYSKIKHLRLDGNRISETSLPPDMYECLRVANEVTLN

SEQ ID NO: 3 - DNA, 20 bp, Artificial Sequence
  Description of Artificial Sequence Synthetic oligonucleotide
tccatgacgttcctgatgct

SEQ ID NO: 4 - DNA, 20 bp, Artificial Sequence
  Description of Artificial Sequence Synthetic primer
tcgagcttgatctctcctat

SEQ ID NO: 5 - DNA, 21 bp, Artificial Sequence
  Description of Artificial Sequence Synthetic primer
tggtcccaggatcttacagaa

SEQ ID NO: 6 - DNA, 18 bp, Artificial Sequence
  Description of Artificial Sequence Synthetic primer
ttgtctcctgcgacttca

SEQ ID NO: 7 - DNA, 20 bp, Artificial Sequence
  Description of Artificial Sequence Synthetic primer
cctgttgctgtagccgtatt

SEQ ID NO: 8 - protein, 12 aa, Artificial Sequence
  Description of Artificial Sequence Synthetic consensus sequence
XLXXLXLXXNXL

SEQ ID NO: 9 - protein, 375 aa, Homo sapiens
MERASCLLLLLLPLVHVSATTPEPCELDDEDFRCVCNFSEPQPDWSEAFQCVSAVEVEIHAGGLNLEPFLKRVDADADPRQYADTVKALRVRRLTVGAAQVPAQLLVGALRVLAYSRLKELTLEDLKITGTMPPLPLEATGLALSSLRLRNVSWATGRSWLAELQQWLKPGLKVLSIAQAHSPAFSCEQVRAFPALTSLDLSDNPGLGERGLMAALCPHKFPAIQNLALRNTGMETPTGVCAALAAAGVQPHSLDLSHNSLRATVNPSAPRCMWSSALNSLNLSFAGLEQVPKGLPAKLRVLDLSCNRLNRAPQPDELPEVDNLTLDGNPFLVPGTALPHEGSMNSGVVPACARSTLSVGVSGTLVLLQGARGFA

(19) United States
(12) Patent Application Publication
(10) Pub. No.: US 2010/0098699 A1
(43) Pub. Date: Apr. 22, 2010

(54) Methods of using LRR superfamily genes and proteins

(75) Inventor: Shukti Chakravarti, Lutherville, MD (US)

Correspondence Address: EDWARDS ANGELL PALMER & DODGE LLP, P.O. BOX 55874, BOSTON, MA, 02205, US

(73) Assignee: The Johns Hopkins University, Baltimore, MD (US)

(21) Appl. No.: 12/313,905

(22) Filed: Nov. 24, 2008

Related U.S. Application Data
(63) Continuation of application No. PCT/US2007/012279, filed on May 23, 2007.
(60) Provisional application No. 60/802,825, filed on May 23, 2006.

(51) Int. Cl. A61K 39/395; A61K 38/16; A61K 38/17; A61K 31/7088

(52) U.S. Cl. 424/141.1; 424/130.1; 514/8; 514/12; 514/44 R; 514/44 A

(57) ABSTRACT

The instant application provides methods and compositions for the treatment of inflammation, for modulating the activity or expression of proinflammatory cytokines, for increasing the rate of wound healing, for treating infection, and for treating septic shock. The invention provides lumican, or modulators thereof, as a therapeutic composition for the treatment of these disorders.

### RELATED APPLICATIONS

This application claims the benefit of U.S. Provisional Application Ser. No. 60/802,825 filed May 23, 2006. The entire contents of the aforementioned provisional application are hereby incorporated herein by reference.

### GOVERNMENT SUPPORT

The following invention was supported at least in part by the a grant from the National Eye Institutes at the National Institutes of Health. Accordingly, the government may have certain rights in the invention.

### BACKGROUND OF THE INVENTION

Innate immunity is the most primitive defense mechanism that the host organism uses to detect and destroy invading pathogens in barrier tissues, without extensive damage to the host barrier. An intense scrutiny of this area has led to an understanding of the elaborate host defense mechanisms that are in place at the cell surface and in the cytoplasm. The current study on the extracellular matrix protein lumican, indicates that there is yet another level of regulation of host defense mechanism, one that is mediated by extracellular Matrix (ECM) proteins classically viewed as structural proteins. A few recent studies have begun to show a role for the ECM in pathogen recognition and/or regulation of innate immune response. Mindin, a member of the spondin family of ECM proteins plays a role in recognition of pathogens and Mindin-deficient mice were hyporesponsive to a variety of pathogen associated molecular patterns (PAMP).

At the cell surface, innate immune response begins with the recognition of PAMPs by the pathogen recognition receptors, the toll-like receptors, on antigen presenting cells like macrophages and dendritic cells. The extracellular domain of these TLRs have leucine-rich repeat (LRR) motifs, found in diverse proteins, from plants to human, such as the ancient resistance proteins in plants (R proteins), Drosophila toll proteins, and mammalian ribonuclease inhibitor. These LRR motifs bind DNA-, RNA- and protein-ligands, including those derived from invading microorganisms. In the cytoplasm a family of cytosolic LRR proteins, the NOD (nucleotide binding oligomerization domain) proteins bind PAMP molecules and promote the innate immune signal. Ultimately, these pathways lead to the phosphorylation of IKKs (IκB kinases), nuclear translocation and the activation of NF-κB, induction of pro-inflammatory cytokines and microbicidal activities. TNFα a pro-inflammatory cytokine prototype is often used to assess the induction of host innate immune response.

Bacterial lipopolysaccharide and lipid A endotoxins activate the TLR4 signaling pathway that triggers the biosynthesis of a variety of inflammation mediators, such as TNFα, IL-1β, IL-6 and other co-stimulatory molecules.

Accordingly, a need exists to better understand the role of extracellular matrix components in the onset and progression of inflammation and infection.

### SUMMARY OF THE INVENTION

The extracellular matrix (ECM) plays an important, but poorly understood role in pathogen associated molecular pattern (PAMP) recognition by host receptors. The instant inventor has discovered for the first time that lumican, an ECM protein with leucine-rich repeats (LRR), is required for bacterial lipopolysaccharide (LPS) sensing by the TLR4-signaling pathway. Mice deficient in lumican produce lower amounts of pro-inflammatory cytokines (TNFα, IL-6), in response to LPS, and are resistant to LPS-mediated septic shock and death. Lum^{-/-} macrophages also fail to induce TNFα in response to LPS, and exogenous recombinant lumican restores this function. In vitro lumican binds LPS that can be competitively removed with CD14, a cell surface LPS-binding protein of the TLR4 pathway. Response to other PAMP is not affected by lumican-deficiency. Lumican is a novel LPS-binding LRR protein of the ECM that specifically enhances LPS sensitivity.

Accordingly, in at least one aspect, the instant invention provides methods of treating inflammation in a subject by administering to the subject an effective amount of a composition that decreases the activity or expression of a LRR superfamily glycoprotein, thereby treating the inflammation in the subject.

In another aspect, the instant invention provides methods of modulating the activity or expression of proinflammatory cytokines in a subject by administering to the subject an effective amount of a composition that decreases the activity or expression of a LRR superfamily glycoprotein, thereby modulating the activity or expression of proinflammatory cytokines in the subject.

In another aspect, the instant invention provides methods of increasing the rate of wound healing comprising, administering to the subject an effective amount of a composition that increases the amount, activity or expression of a LRR superfamily glycoprotein, thereby increasing the rate of wound healing in a subject. In one embodiment, the LRR superfamily glycoprotein is lumican.

In another aspect, the instant invention provides methods of treating a subject having a bacterial infection by administering to the subject an effective amount of a composition that decreases the activity or expression of a LRR superfamily glycoprotein, thereby treating the subject.

In another aspect, the instant invention provides methods of treating a subject having septic shock by administering to the subject an effective amount of a composition that decreases the activity or expression of a LRR superfamily glycoprotein, thereby treating the subject.

In specific embodiments, the LRR superfamily glycoprotein binds to LPS. In further specific embodiments, the LRR superfamily glycoprotein is lumican.

In certain embodiments, the composition comprises a glycoprotein, fragment thereof, peptide mimetic, antibody, small molecule, antisense RNA, siRNA, shRNA, ribozyme or aptamer.

In certain embodiments, the glycoprotein is lumican, e.g., the polypeptide set forth as SEQ OD NO:2, or a fragment thereof. In a related embodiment, the lumican fragment comprises the sequence XL²XXL⁵XL⁷XXN¹⁰XL.

In one embodiment, the composition comprises an antibody is a blocking antibody, monoclonal antibody, polyclonal antibody, or fragment thereof. In related embodiments, the antibody is human or humanized.

In another aspect, the instant invention provides pharmaceutical compositions comprising a lumican modulator. In a related embodiment, the modulator is a modulator of lumican as set forth as SEQ ID NO:2, or a fragment thereof. In another related embodiment, fragments of lumican comprise the sequence XL²XXL⁵XL⁷XXN¹⁰XL (SEQ ID NO:8).

In another aspect, the instant invention provides pharmaceutical compositions comprising an siRNA, antisense RNA, or shRNA specific for a lumican encoding nucleic acid.

In another aspect, the invention provides kits for the treatment of inflammation, for wound healing, for modulating the expression of proinflammatory cytokines, for the treatment of a bacterial infection, or for the treatment of septic shock comprising an agent or composition that modulates the expression or activity of lumican and instructions for use. In one embodiment, the agent is a glycoprotein, e.g., a LRR superfamily glycoprotein or a fragment thereof peptide mimetic, antibody, small molecule, an LRR superfamily nucleic acid molecule, antisense RNA, siRNA, shRNA, ribozyme or aptamer.

In another embodiment, the glycoprotein is lumican, e.g., the glycoprotein set forth as SEQ ID NO:2, or a fragment thereof. In another related embodiment, fragments of lumican comprise the sequence XL²XXL⁵XL⁷XXN¹⁰XL (SEQ ID NO:8).

In one embodiment, the kit comprises an antibody, e.g., a blocking antibody, a monoclonal antibody, a polyclonal antibody, or fragment thereof. In related embodiments, the antibody is human or humanized.

In another embodiment, the kit comprises an siRNA, antisense RNA, or shRNA that binds to a LRR superfamily nucleic acid molecule, e.g., a lumican nucleic acid molecule.

### DESCRIPTION OF THE DRAWINGS

FIGS. 1A-C demonstrate that Lum^{-/-} mice are hypo-responsive to LPS. A single dose of 16.7 μg/g body weight of Salmonella typhimurium LPS was administered by intraperitoneal injection. (a) Mice 24 h after LPs treatment. The Lum^{+/+} mice showed visible signs of sickness, while the Lum^{-/-} mice appeared healthy. (b) Percent survival in response to LPS-mediated septic shock. Age and gender matched mice were treated with LPS (n=8) or saline (nom) as control (not shown). One of six experiments shown here demonstrates higher survival of Lum^{-/-} mice. Five similar experiments conducted with E. coli LPS yielded similar survival trends (not shown). (c) Hematoxylin and eosin staining of sections of the spleen from mice challenged with LPS showed signs of immune response in the Lum^{+/+}, but little or no signs of immune response in the LPS-treated Lum^{-/-} mice.

FIGS. 2A-B demonstrate lower induction of serum cytokines in Lum^{-/-} mice compared to Lum^{+/+} mice after a systemic challenge of LPS. The serum was harvested 32 hours after one intraperitoneal injection of 16.7 μg/g body weight of LPS. (a) TNFα was measured by ELISA in littermate animals treated with LPS (n=7) or saline as a control (n=5). The result is presented as mean±1 s.d. * Significant difference (p ≦0.05) in TNFα induction in Lum^{+/+} and Lum^{-/-} mice. (b) Quantitative measurements were obtained for multiple cytokines in age and gender matched mice (n=4 per treatment) using the Beadlyte® Mouse Multi-Cytokine Flex Kit. The results shown are mean±1 s.d. LPS-S and LPS-E are S. typhimurium LPS and E. coli LPS, respectively.

FIGS. 3A-B demonstrate reduced induction of TNFα and IL-6 in Lum^{-/-} peritoneal macrophage cultures. Lum^{+/+} and Lum^{-/-} littermate animals were given an intraperitoneal injection of 4% thioglycolate to elicit macrophages. The peritoneal lavage macrophages were collected 4 days later and cultured for 24 hours. The macrophages were treated with E. coli LPS at 10 ng/ml followed by measurements of cytokine measurement in the medium by ELISA. (a) TNFα ELISA. The LPS treated Lum^{-/-} macrophages showed consistently lower induction of TNFα. (b) IL-6 ELISA. The LPS treated Lum^{-/-} macrophages showed consistently lower induction of IL-6. The mean of three samples±1 s.d are shown. * Significant difference (p≦0.01) in cytokine between Lum^{+/+} and Lum^{-/-} mice.

FIGS. 4A-B demonstrate that recombinant lumican (rLum) rescues LPS-mediated TNFα induction. (a) Addition of rLum increased LPS-mediated induction of TNFα in the Lum^{-/-} macrophage cultures. rLum alone did not induce TNFα (b) Ovalbumin, used as a control protein, in a similar rescue experiment failed to restore normal TNFα induction in the Lum^{-/-} macrophages. The mean±1 s.d. of three samples are shown for each experiment.

FIG. 5 demonstrates that Lum^{-/-} macrophages are specifically impaired in responding to LPS and not to other PAMPs. Induction of TNFα was measured by ELISA in elicited peritoneal macrophages from Lum^{+/+} and Lum^{-/-} littermates. The macrophages were treated with either 10 ng/ml LPS, 10 μg/ml PGN (peptidoglycan), 10 ng/ml MDP (muramyl dipeptide), 1 μM CpG-DNA (TCCATGACGTTCCTGATGCT (SEQ ID NO:3)), 10 μg/ml Poly I:C (polyinosinic-polycytidylic acid) for 4 h. The corresponding pathogen recognition proteins for each of the PAMPs are shown below the graph. TNFα induction in response to all the PAMPS was comparable in Lum^{+/+} and Lum^{-/-}, except that in response to LPS, where it was lower in the Lum^{-/-} macrophages. The mean±1 s.d. of three samples is shown.

FIG. 6 depicts NF-κB activation in response to LPS treatment was delayed in Lum^{-/-} bone marrow macrophages. NF-κB DNA binding activity was measured by electrophoretic mobility gel shift assays in nuclear extracts of Lum^{+/+} and Lum^{-/-} littermate bone marrow derived macrophages treated with LPS in culture. NF-κB was induced maximally after 10 min of LPS treatment in the wild type; an almost similar extent of activation was achieved after 20 minutes of LPS in the Lum^{-/-} macrophages.

FIGS. 7A-B demonstrate the response to live bacteria was not impaired by lumican-deficiency. (a) Bacterial infection after an intraperitoneal injection of live S. typhimurium, was comparable in the Lum^{-/-} and Lum^{+/+} mice as indicated by bacterial yield, measured as colony forming units (CFU) from spleen and liver. (b) TNFα induction in the serum, measured by ELISA, showed no difference between Lum^{-/-} and Lum^{+/+} mice challenged with S. typhimurium. N=5 per strain challenged with bacteria or n=2 per genotype treated with saline as control. Measurements are mean±1 s.d.

FIGS. 8A-C demonstrate that lumican is induced during innate immune response. Lum^{+/+} mouse embryonic fibroblast cultures were treated for 24 h with (a) LPS, (b), IL-1β (c) TGFβ and lumican mRNA measured in the total RNA by quantitative RT-PCR. For each sample n=3, and mean±1 s.d. is shown. Lumican transcript increased in cells treated with LPS and IL-1β, but was suppressed by TGFβ.

FIGS. 9A-C demonstrate low expression of lumican in macrophages but lumican is associated with macrophage cell surfaces. (a) Peritoneal macrophages from Lum^{+/+} mice were treated with 10 ng/ml of LPS for different periods of time as indicated and lumican expression was quantified by qRT-PCR from total RNA extracts of the cells and normalized to Gapdh expression. (b) A subset of macrophages shows lumican on their surface. Peritoneal macrophages from Lum^{+/+} mice were immunostained with F4/80, a macrophage specific antibody and a secondary goat anti-rat Alexa Fluor 568 (top panel) and anti-lumican followed by anti-rabbit texas red as a secondary antibody (middle panel) and stained with 1% Hoechst dye for nuclear staining of all the cells (bottom panel). Bar=20 μm. (c) Exogenous recombinant lumican (rLum) was able to bind to Lum^{-/-} macrophages. Lum^{-/-} macrophages incubated with rLum were extracted and rLum bound to the macrophage surface detected by ELISA. BSA-blocked wells were used to assess background staining, and wells coated with 1 μg/well rLum were used to determine maximum immunopositive staining. Lum^{-/-} macrophages, not treated with rLum (untreated control), showed immunostaining that was equivalent to the background, while the rLum-treated cells showed cell extract-dose dependent increase in absorbance. The results are mean±1 s.d. of three wells per sample. *=Significant difference (p<0.05) between rLum treated and untreated macrophage cell extract.

FIGS. 10A-C demonstrate that lumican binds LPS. (a) Binding of LPS to recombinant lumican (rLum) was tested in a solid-phase binding assay. The wells were coated with 100 ng/well of rLum, and coated and uncoated wells were blocked with 3% bovine serum albumin in PBS for 2 hours and increasing doses of FITC-LPS was incubated in the wells, washed and FITC-LPS retained in the wells were determined by fluorescence. Wells coated with rLum show bound and retained FITC-LPS. (b) FITC-LPS bound to rLum could be competed out with a 10-fold excess of unlabeled LPS. (c) Soluble CD14 was added to the wells at the time of adding FITC-LPS and bound FITC-LPS measured after three washes.

FIG. 11 depicts the amino acid sequence comparison between human lumican and CD14. In yellow highlights are LPS binding sites in CD14. Highlighted in gray are synthetic peptide sequences. Sites for proposed mutations are marked with an asterisk.

FIGS. 12A-B set for the nucleic acid and polypeptide sequence of lumican, respectively.

### DETAILED DESCRIPTION OF THE INVENTION

The various embodiments of the instant invention are based on the discovery by the inventor that lumican, an extra cellular matrix protein with leucine-rich repeats (LRR), is required for bacterial lipopolysaccharide (LPS) sensing by the TLR4-signaling pathway, and that mice deficient in lumican produce lower amounts of pro-inflammatory cytokines (TNFα, IL-6), in response to LPS, and are resistant to LPS-mediated septic shock and death.

### Recombinant Expression Vectors and Host Cells Used in the Methods of the Invention

The methods of the invention (e.g., the screening assays described herein) include the use of vectors, preferably expression vectors, containing a nucleic acid encoding a LRR superfamily glycoprotein (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors to be used in the methods of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel (1990) Methods Enzymol. 185:3-7. Regulatory sequences include those which direct constitutive expression of a nucleotide sequence in many types of host cells and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein.

The recombinant expression vectors to be used in the methods of the invention can be designed for expression of LRR superfamily glycoproteins in prokaryotic or eukaryotic cells. For example, proteins can be expressed in bacterial cells such as E. coli, insect cells (using baculovirus expression vectors), yeast cells, or mammalian cells. Suitable host cells are discussed further in Goeddel (1990) supra. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

In another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J. et al., Molecular Cloning: A Laboratory Manual. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid).

The methods of the invention may further use a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to LRR superfamily mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific, or cell type specific expression of antisense RNA. The antisense expression vector can be in the form of a recombinant plasmid, phagemid, or attenuated virus in which antisense nucleic acids are produced under the control of a high efficiency regulatory region, the activity of which can be determined by the cell type into which the vector is introduced. For a discussion of the regulation of gene expression using antisense genes, see Weintraub, H. et al., Antisense RNA as a molecular tool for genetic analysis, Reviews—Trends in Genetics, Vol. 1(1) 1986.

Another aspect of the invention pertains to the use of host cells into which a LRR superfamily nucleic acid molecule of the invention is introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

A host cell used in the methods of the invention, such as a prokaryotic or eukaryotic host cell in culture, can be used to produce (i.e., express) a LRR superfamily glycoprotein. Accordingly, the invention further provides methods for producing a LRR superfamily glycoprotein using the host cells of the invention. In one embodiment, the method comprises culturing the host cell of the invention (into which a recombinant expression vector encoding a LRR superfamily glycoprotein has been introduced) in a suitable medium such that a protein is produced. In another embodiment, the method further comprises isolating a protein from the medium or the host cell.

The nucleic acid molecules used in the methods of the invention can be inserted into vectors and used as gene therapy vectors. Gene therapy vectors can be delivered to a subject by, for example, intravenous injection, local administration (see U.S. Pat. No. 5,328,470) or by stereotactic injection (see, e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

Another aspect of the invention pertains to the use of isolated nucleic acid molecules which are antisense to the nucleotide sequence of SEQ ID NO:1. An “antisense” nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire coding strand, or to only a portion thereof. In one embodiment, an antisense nucleic acid molecule is antisense to a “coding region” of the coding strand of a nucleotide sequence encoding a LRR superfamily glycoprotein, e.g., lumican. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding a LRR superfamily glycoproteins. The term “noncoding region” refers to 5′ and 3′ sequences which flank the coding region that are not translated into amino acids (also referred to as 5′ and 3′ untranslated regions).

Given the coding strand sequences encoding a LRR superfamily glycoproteins disclosed herein, antisense nucleic acids of the invention can be designed according to the rules of Watson and Crick base pairing. The antisense nucleic acid molecule can be complementary to the entire coding region of the mRNA, but more preferably is an oligonucleotide which is antisense to only a portion of the coding or noncoding region of the mRNA. For example, the antisense oligonucleotide can be complementary to the region surrounding the translation start site of 46566 mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomet-hyluracil, dihydrouracil, beta-D-galactosylqueosine, inosine, N6-isopentenyladenine, 1-methylguanine, 1-methyl inosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopenten-yladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules used in the methods of the invention are typically administered to a subject or generated in situ such that they hybridize with or bind to cellular mRNA and/or genomic DNA encoding a LRR superfamily glycoprotein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention include direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule used in the methods of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid used in the methods of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haseloff and Gerlach (1988) Nature 334:585-591)) can be used to catalytically cleave 46566 mRNA transcripts to thereby inhibit translation of the mRNA. A ribozyme having specificity for a 46566-encoding nucleic acid can be designed based upon the nucleotide sequence of a 46566 cDNA disclosed herein (i.e., SEQ ID NO:1). For example, a derivative of a Tetrahymena L-19 WS RNA can be constructed in which the nucleotide sequence of the active site is complementary to the nucleotide sequence to be cleaved in a 46566-encoding mRNA. See, e.g., Cech et al. U.S. Pat. No. 4,987,071; and Cech et al. U.S. Pat. No. 5,116,742. Alternatively, 46566 mRNA can be used to select a catalytic RNA having a specific ribonuclease activity from a pool of RNA molecules. See, e.g., Bartel, D. and Szostak, J. W. (1993) Science 261:1411-1418.

Alternatively, LRR superfamily gene expression can be inhibited by targeting nucleotide sequences complementary to the regulatory region of the gene (e.g., the promoter and/or enhancers) to form triple helical structures that prevent transcription of the 46566 gene in target cells. See generally, Helene, C. (1991) Anticancer Drug Des. 6(6): 569-84; Helene, C. et al. (1992) Ann. N.Y. Acad. Sci. 660:27-36; and Maher, L. J. (1992) Bioessays 14(12):807-15.

In yet another embodiment, the nucleic acid molecules used in the methods of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup, B. and Nielsen, P. E. (1996) Bioorg. Med. Chem. 4(1):5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. and Nielsen (1996) supra and Perry-O'Keefe et al. (1996) Proc. Natl. Acad. Sci. USA 93:14670-675.

PNAs of LRR superfamily nucleic acid molecules can be used in the therapeutic and diagnostic applications described herein. For example, PNAs can be used as antisense or antigen agents for sequence-specific modulation of gene expression by, for example, inducing transcription or translation arrest or inhibiting replication. PNAs of the nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup and Nielsen (1996) supra)); or as probes or primers for DNA sequencing or hybridization (Hyrup and Nielsen (1996) supra; Perry-O'Keefe et al. (1996) supra).

In another embodiment, PNAs can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of 46566 nucleic acid molecules can be generated which may combine the advantageous properties of PNA and DNA. Such chimeras allow DNA recognition enzymes, (e.g., RNAse H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup and Nielsen (1996) supra). The synthesis of PNA-DNA chimeras can be performed as described in Hyrup and Nielsen (1996) supra and Finn P. J. et al. (1996) Nucleic Acids Res. 24 (17): 3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a between the PNA and the 5′ end of DNA (Mag, M. et al. (1989) Nucleic Acids Res. 17: 5973-88). PNA monomers are then coupled in a stepwise manner to produce a chimeric molecule with a 5′ PNA segment and a 3′ DNA segment (Finn et al. (1996) supra). Alternatively, chimeric molecules can be synthesized with a 5′ DNA segment and a 3′ PNA segment (Peterser, K. H. et al. (1975) Bioorganic Med. Chem. Lett. 5: 1119-11124).

In other embodiments, the oligonucleotide used in the methods of the invention may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. WO88/09810) or the blood-brain barrier (see, e.g., PCT Publication No. WO89/10134). In addition, oligonucleotides can be modified with hybridization-triggered cleavage agents (See, e.g., Krol et al. (1988) Biotechniques 6:958-976) or intercalating agents. (See, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

### Isolated Proteins and Antibodies Used in the Methods of the Invention

The methods of the invention include the use of isolated LRR superfamily glycoproteins, and biologically active portions thereof, as well as polypeptide fragments suitable for use as immunogens to raise anti-LRR superfamily glycoprotein antibodies. In one embodiment, native proteins can be isolated from cells or tissue sources by an appropriate purification scheme using standard protein purification techniques. In another embodiment, proteins are produced by recombinant DNA techniques. Alternative to recombinant expression, a protein or polypeptide can be synthesized chemically using standard peptide synthesis techniques. In one embodiment the LRR superfamily glycoprotein is lumican.

As used herein, “LRR superfamily glycoproteins” refers to a related group of extracellular matrix proteins. The LRR superfamily includes, among other proteins, decorin, biglycan, fibromodulin, lumican, Epiphycan/PG-Lb and mimecan/osteoglycin (see, Iozzo, R. V. (1999) J Biol Chem 274, 18843-18846).

As used herein, “lumican” refers to a polypeptide set forth as SEQ ID NO:2 (NCBI Accession No: AAP35353) or a nucleic acid set forth as SEQ ID NO:1 (NCBI Accession No. BT006707). Lumican is a proteoglycan of the extracellular matrix, and one of approximately 12 related proteoglycans of the LRR protein superfamily (Iozzo, R. V. (1999) J Biol Chem 274, 18843-18846). It is expressed in a variety of stromal mesenchymal ECM of barrier tissues, such as the interstitial extracellular matrix of the skin, the cornea, the intestine and other connective tissues. In addition to this well-established structural role, these proteins also regulate cellular proliferation, and apoptosis. Lumican belongs to the small leucine-rich proteoglycan (SLRP) family (class II subfamily) and contains 12 LRR (leucine-rich) repeats having the consensus sequence XL²XXL⁵XL⁷XXN¹⁰XL, where L represents leucine residues, N, a conserved asparagine at position 10, and X represents any amino acid. Included in the methods and compositions of the invention are lumican molecules from species other than human. For example, murine lumican can be used in the methods and compositions of the instant invention.

As used herein, a “biologically active portion” of a LRR superfamily glycoprotein includes a fragment of a LRR superfamily glycoprotein having a native activity. Biologically active portions of a LRR superfamily glycoprotein include peptides comprising amino acid sequences sufficiently identical to or derived from the amino acid sequence of the LRR superfamily glycoprotein such as lumican, e.g., the amino acid sequence shown in SEQ ID NO:2, which include fewer amino acids than the full length protein, and exhibit at least one activity of the protein. Typically, biologically active portions comprise a domain or motif with at least one activity of the LRR superfamily glycoprotein. A biologically active portion of a LRR superfamily glycoprotein can be a polypeptide which is, for example, 25, 50, 75, 100, 125, 150, 175, 200, 250, 300 or more amino acids in length.

In a preferred embodiment, the LRR superfamily glycoprotein used in the methods of the invention is lumican and has an amino acid sequence shown in SEQ ID NO:2. In other embodiments, the protein is substantially identical to SEQ ID NO:2, and retains the functional activity of the protein of SEQ ID NO:2, yet differs in amino acid sequence due to natural allelic variation or mutagenesis. Accordingly, in another embodiment, the protein used in the methods of the invention is a protein which comprises an amino acid sequence at least about 50%, 55%, 60%, 65%, 70%, 75%, 80%, 85%, 90%, 95%, 96%, 97%, 98%, 99%, 99.1%, 99.2%, 99.3%, 99.4%, 99.5%, 99.6%, 993%, 99.8%, 99.9% or more identical to SEQ ID NO:2, or a biological fragment thereof.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-identical sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. 48:444-453 (1970)) algorithm which has been incorporated into the GAP program in the GCG software package (available at http://www.gcg.com), using either a Blosum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (Comput. Appl. Biosci. 4:11-17 (1988)) which has been incorporated into the ALIGN program (version 2.0 or 2.0 U), using a PAM120 weight residue table, a gap length penalty of 12 and a gap penalty of 4.

The methods of the invention may also use chimeric or fusion proteins. As used herein, a “chimeric protein” or “fusion protein” comprises a polypeptide operatively linked to a non-LRR superfamily glycoprotein.

In addition, libraries of fragments of a LRR superfamily glycoprotein coding sequence can be used to generate a variegated population of LRR superfamily glycoprotein fragments for screening and subsequent selection of variants of a LRR superfamily glycoprotein. In one embodiment, a library of coding sequence fragments can be generated by treating a double stranded PCR fragment of a coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the protein.

The methods of the present invention further include the use of anti-LRR superfamily glycoprotein antibodies. An isolated LRR superfamily glycoprotein, or a portion or fragment thereof, can be used as an immunogen to generate antibodies that bind a LRR superfamily glycoprotein using standard techniques for polyclonal and monoclonal antibody preparation. A full-length protein can be used or, alternatively, antigenic peptide fragments of LRR superfamily glycoprotein, e.g., can be used as immunogens. The antigenic peptide of 46566 comprises at least 8 amino acid residues of the amino acid sequence shown in SEQ ID NO:2 and encompasses an epitope of 46566 such that an antibody raised against the peptide forms a specific immune complex with the 46566 protein. Preferably, the antigenic peptide comprises at least 10 amino acid residues, more preferably at least 15 amino acid residues, even more preferably at least 20 amino acid residues, and most preferably at least 30 amino acid residues.

Preferred epitopes encompassed by the antigenic peptide are regions of LRR superfamily glycoproteins that are located on the surface of the protein, e.g., hydrophilic regions, as well as regions with high antigenicity.

A LRR superfamily glycoprotein immunogen is typically used to prepare antibodies by immunizing a suitable subject, (e.g., rabbit, goat, mouse, or other mammal) with the immunogen. An appropriate immunogenic preparation can contain, for example, recombinantly expressed protein or a chemically synthesized polypeptide. The preparation can further include an adjuvant, such as Freund's complete or incomplete adjuvant, or similar immunostimulatory agent. Immunization of a suitable subject with an immunogenic LRR superfamily glycoprotein preparation induces a polyclonal anti-LRR superfamily glycoprotein antibody response.

The term “antibody” as used herein refers to immunoglobulin molecules and immunologically active portions of immunoglobulin molecules, i.e., molecules that contain an antigen binding site which specifically binds (immunoreacts with) an antigen, such as a lumican. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind LRR superfamily glycoprotein molecules. The term “monoclonal antibody” or “monoclonal antibody composition”, as used herein, refers to a population of antibody molecules that contain only one species of an antigen binding site capable of immunoreacting with a particular epitope of a LRR superfamily glycoprotein. A monoclonal antibody composition thus typically displays a single binding affinity for a particular LRR superfamily glycoprotein with which it immunoreacts.

Polyclonal anti-LRR superfamily glycoprotein antibodies can be prepared as described above by immunizing a suitable subject with a LRR superfamily glycoprotein immunogen. The anti-LRR superfamily glycoprotein antibody titer in the immunized subject can be monitored over time by standard techniques, such as with an enzyme linked immunosorbent assay (ELISA) using immobilized LRR superfamily glycoprotein. If desired, the antibody molecules directed against 46566 can be isolated from the mammal (e.g., from the blood) and further purified by well known techniques, such as protein A chromatography to obtain the IgG fraction. At an appropriate time after immunization, e.g., when the anti-LRR superfamily glycoprotein antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem. 255:4980-83; Yeh et al. (1976) Proc. Natl. Acad. Sci. USA 76:2927-31; and Yeh et al. (1982) Int. J. Cancer 29:269-75), the more recent human B cell hybridoma technique (Kozbor et al. (1983) Immunol. Today 4:72), the EBV-hybridoma technique (Cole et al. (1985) Monoclonal Antibodies and Cancer Therapy, Alan R. Liss, Inc., pp. 77-96) or trioma techniques. The technology for producing monoclonal antibody hybridomas is well known (see generally Kenneth, R. H. in Monoclonal Antibodies: A New Dimension In Biological Analyses, Plenum Publishing Corp., New York, N.Y. (1980); Lerner, E. A. (1981) Yale J. Biol. Med. 54:387-402; Gefter, M. L. et al. (1977) Somat. Cell Genet. 3:231-36). Briefly, an immortal cell line (typically a myeloma) is fused to lymphocytes (typically splenocytes) from a mammal immunized with a 46566 immunogen as described above, and the culture supernatants of the resulting hybridoma cells are screened to identify a hybridoma producing a monoclonal antibody that binds a LRR superfamily glycoprotein, e.g., lumican.

Any of the many well known protocols used for fusing lymphocytes and immortalized cell lines can be applied for the purpose of generating an anti-LRR superfamily glycoprotein monoclonal antibody (see, e.g., G. Galfre et al. (1977) Nature 266:55052; Gefter at al. (1977) supra; Lerner (1981) supra; and Kenneth (1980) supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind the LRR superfamily glycoprotein, e.g., using a standard ELISA assay.

Alternative to preparing monoclonal antibody-secreting hybridomas, a monoclonal anti-LRR superfamily glycoprotein antibody can be identified and isolated by screening a recombinant combinatorial immunoglobulin library (e.g., an antibody phage display library) with LRR superfamily glycoprotein to thereby isolate immunoglobulin library members that bind 46566. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower at al. PCT International Publication No. WO 91/17271; Winter at al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Blip/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734; Hawkins et al. (1992) J. Mol. Biol. 226:889-896; Clarkson et al. (1991) Nature 352:624-628; Gram et al. (1992) Proc. Natl. Acad. Sci. USA 89:3576-3580; Garrard et al. (1991) Biotechnology (NY) 9:1373-1377; Hoogenboom et al. (1991) Nucleic Acids Res. 19:4133-4137; Barbas et al. (1991) Proc. Natl. Acad. Sci. USA 88:7978-7982; and McCafferty et al. (1990) Nature 348:552-554.

Additionally, recombinant anti-LRR superfamily glycoprotein antibodies, such as chimeric and humanized monoclonal antibodies, comprising both human and non-human portions, which can be made using standard recombinant DNA techniques, are within the scope of the methods of the invention. Such chimeric and humanized monoclonal antibodies can be produced by recombinant DNA techniques known in the art, for example using methods described in Robinson et al. international Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immunol. 13.9:3521-3526; Sun et al. (1987) Proc. Natl. Acad. Sci. USA 84:214-218; Nishimura et al. (1987) Cancer Res. 47:999-1005; Wood et al. (1985) Nature 314:446-449; Shaw et al. (1988) J. Natl. Cancer Inst. 80:1553-1559; Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyen et al. (1988) Science 239:1534; and Beidler et al. (1988) J. Immunol. 141:4053-4060.

An anti-LRR superfamily glycoprotein antibody can be used to detect LRR superfamily glycoproteins (e.g., in a cellular lysate or cell supernatant) in order to evaluate the abundance and pattern of expression of the LRR superfamily glycoprotein protein. Anti-LRR superfamily glycoprotein antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, .beta.-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

As used interchangeably herein, the terms “activity of a LRR superfamily glycoprotein,” “biological activity of a LRR superfamily glycoprotein” or “functional activity of a LRR superfamily glycoprotein,” include an activity exerted by a of a LRR superfamily glycoprotein, polypeptide or nucleic acid molecule, e.g., a lumican polypeptide or nucleic acid molecule as determined in vivo, or in vitro, according to standard techniques. LRR superfamily glycoprotein activity can be a direct activity.

### Screening Assays

The invention provides methods (also referred to herein as “screening assays”) for identifying modulators, i.e., candidate or test compounds or agents (e.g., polypeptides, peptides, peptidomimetics, nucleic acid molecules, small molecules, antibodies, ribozymes, or antisense molecules) which bind to LRR superfamily glycoproteins, e.g., lumican, have a stimulatory or inhibitory effect on the LRR superfamily glycoprotein's expression or activity, or have a stimulatory or inhibitory effect on the expression or activity of a LRR superfamily glycoprotein target molecule. Compounds identified using the assays described herein may be useful for treating, for example, inflammation.

Candidate/test compounds include, for example, 1) peptides such as soluble peptides, including Ig-tailed fusion peptides and members of random peptide libraries (see, e.g., Lam, K. S. et al. (1991) Nature 354:82-84; Houghten, R. et al. (1991) Nature 354:84-86) and combinatorial chemistry-derived molecular libraries made of D- and/or L-configuration amino acids; 2) phosphopeptides (e.g., members of random and partially degenerate, directed phosphopeptide libraries, see, e.g., Songyang, Z. et al. (1993) Cell 72:767-778); 3) antibodies (e.g., polyclonal, monoclonal, humanized, anti-idiotypic, chimeric, and single chain antibodies as well as Fab, F(ab).sub.2, Fab expression library fragments, and epitope-binding fragments of antibodies); and 4) small organic and inorganic molecules (e.g., molecules obtained from combinatorial and natural product libraries).

The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in DeWitt et al. (1993) Proc. Natl. Acad. Sci. USA 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2661; and Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc. Natl. Acad. Sci. USA 89:1865-1869) or phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310; Ladner supra.).

In one aspect, an assay is a cell-based assay in which a cell which expresses a LRR superfamily glycoprotein or biologically active portion thereof is contacted with a test compound and the ability of the test compound to modulate the LRR superfamily glycoprotein activity is determined. In a preferred embodiment, the biologically active portion of the LRR superfamily glycoprotein includes a domain or motif that can modulate inflammation. Determining the ability of the test compound to modulate the LRR superfamily glycoprotein activity can be accomplished by monitoring, for example, modulation of inflammation. The cell, for example, can be of mammalian origin.

The ability of the test compound to modulate a LRR superfamily glycoprotein binding to a substrate can also be determined. Determining the ability of the test compound to modulate binding to a substrate can be accomplished, for example, by coupling the substrate with a radioisotope, fluorescent, or enzymatic label such that binding of the substrate to the protein can be determined by detecting the labeled substrate in a complex. Alternatively, the protein could be coupled with a radioisotope or enzymatic label to monitor the ability of a test compound to modulate binding to a substrate in a complex. Determining the ability of the test compound to bind the protein can be accomplished, for example, by coupling the compound with a radioisotope or enzymatic label such that binding of the compound to the protein can be determined by detecting the labeled compound in a complex. For example, substrates can be labeled, either directly or indirectly, and the radioisotope detected by direct counting of radioemission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a compound to interest with a LRR superfamily glycoprotein without the labeling of any of the interactants. For example, a microphysiometer can be used to detect the interaction of a compound with 46566 without the labeling of either the compound or the 46566 (McConnell, H. M. et al. (1992) Science 257:1906-1912). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and 46566.

Assays that may be used to identify compounds that modulate 46566 activity also include assays that test for the ability of a compound to modulate inflammation. The ability of a test compound to modulate inflammation can be measured by its ability to modulate inflammation of the tissues surrounding the site of injury.

In yet another embodiment, an assay of the present invention is a cell-free assay in which a LRR superfamily glycoprotein or biologically active portion thereof is contacted with a test compound and the ability of the test compound to bind to or to modulate (e.g., stimulate or inhibit) the activity of the LRR superfamily glycoprotein or biologically active portion thereof is determined. Preferred biologically active portions of the proteins to be used in assays of the present invention include fragments that participate in interactions with non-LRR superfamily molecules, e.g., fragments with high surface probability scores. Alternatively, biologically active fragments may include fragments comprising one or more LLR. Binding of the test compound to the protein can be determined either directly or indirectly as described above. Determining the ability of the protein to bind to a test compound can also be accomplished using a technology such as real-time Biomolecular Interaction Analysis (BIA) (Sjolander, S. and Urbaniczky, C. (1991) Anal. Chem. 63:2338-2345; Szabo et al. (1995) Curr. Opin. Struct. Biol. 5:699-705). As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In yet another embodiment, the cell-free assay involves contacting a protein or biologically active portion thereof with a known compound which binds the protein to form an assay mixture, contacting the assay mixture with a test compound, and determining the ability of the test compound to interact with the protein, wherein determining the ability of the test compound to interact with the protein comprises determining the ability of the protein to preferentially bind to or modulate the activity of a target molecule.

In more than one embodiment of the above assay methods of the present invention, it may be desirable to immobilize either the LRR superfamily glycoprotein or a target molecule to facilitate separation of complexed from uncomplexed forms of one or both of the proteins, as well as to accommodate automation of the assay. Binding of a test compound to a protein, or interaction of a protein with a target molecule in the presence and absence of a test compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix.

In yet another aspect of the invention, the LRR superfamily glycoprotein or fragments thereof can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300) to identify other proteins which bind to or interact with the LRR superfamily glycoprotein.

In another aspect, the invention pertains to a combination of two or more of the assays described herein. For example, a modulating agent can be identified using a cell-based or a cell-free assay, and the ability of the agent to modulate the activity of a LRR superfamily glycoprotein can be confirmed in vivo, e.g., in an animal such as an animal model for inflammation.

In one aspect, the invention provides a method for preventing infection septic shock or inflammation, by administering to the subject an agent which modulates the expression or activity of LRR superfamily glycoprotein expression or activity in a cell. Subjects at risk for developing a inflammation or infection can be identified by, for example, any or a combination of the diagnostic or prognostic assays described herein. Administration of a prophylactic agent can occur prior to the manifestation of symptoms such that a inflammation or infection is prevented or, alternatively, delayed in its progression. The appropriate agent can be determined based on screening assays described herein.

Another aspect of the invention pertains to methods for treating a subject suffering from a inflammation, infection, septic shock, having a wound, or modulating activity or expression of proinflammatory cytokines. These methods involve administering to a subject an agent which modulates the expression or activity of a LRR superfamily glycoprotein e.g., lumican (e.g., an agent identified by a screening assay described herein), or a combination of such agents.

The agents which modulate the activity or expression of a LRR superfamily glycoprotein or nucleic acid molecule can be administered to a subject using pharmaceutical compositions suitable for such administration. Such compositions typically comprise the agent (e.g., nucleic acid molecule, protein, or antibody) and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition used in the therapeutic methods of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the agent that modulates the activity or expression of a LRR superfamily glycoprotein or nucleic acid in the required amount in an appropriate solvent with one or a combination of ingredients enumerated above, as required, followed by filtered sterilization. Generally, dispersions are prepared by incorporating the active compound into a sterile vehicle which contains abasic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The agents that modulate the activity or expression of a LRR superfamily glycoprotein can also be prepared in the form of suppositories (e.g., with conventional suppository bases such as cocoa butter and other glycerides) or retention enemas for rectal delivery.

In one embodiment, the agents that modulate the activity or expression of LRR superfamily glycoproteins are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the agent used and the particular therapeutic effect to be achieved, and the limitations inherent in the art of compounding such an agent for the treatment of subjects.

Toxicity and therapeutic efficacy of such agents can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and can be expressed as the ratio LD50/ED50. Agents which exhibit large therapeutic indices are preferred. While agents that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage for use in humans. The dosage of such modulating agents lies preferably within a range of circulating concentrations that include the ED50 with little or no toxicity. The dosage may vary within this range depending upon the dosage form employed and the route of administration utilized. For any agent used in the therapeutic methods of the invention, the therapeutically effective dose can be estimated initially from cell culture assays. A dose may be formulated in animal models to achieve a circulating plasma concentration range that includes the 1050 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of an agent (i.e., an effective dosage) ranges from about 0.001 to 30 mg/kg, about 0.01 to 25 mg/kg, about 0.1 to 20 mg/kg, and about 1 to 10 mg/kg, 2 to 9 mg/kg, 3 to 8 mg/kg, 4 to 7 mg/kg, or 5 to 6 mg/kg. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

In a preferred example, a subject is treated with antibody, protein, or polypeptide in the range of between about 0.1 to 20 mg/kg pain, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic assays as described herein.

The present invention encompasses agents which modulate the expression or activity of LRR superfamily glycoproteins or nucleic acid molecules. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention. Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram). It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a 46566 polypeptide or nucleic acid molecule, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, pain, general health, gender, and diet of the subject, the time of administration, the route of administration, the rate of excretion, any drug combination, and the degree of expression or activity to be modulated.

Further, the agent may be an antibody (or fragment thereof). In one embodiment, the antibody is a blocking antibody. In another embodiment, the antibody can be conjugated to a therapeutic moiety

The conjugates of the invention can be used for modifying a given biological response, the drug moiety is not to be construed as limited to classical chemical therapeutic agents. For example, the drug moiety may be a protein or polypeptide possessing a desired biological activity.

Techniques for conjugating such therapeutic moiety to antibodies are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982). Alternatively, an antibody can be conjugated to a second antibody to form an antibody heteroconjugate as described by Segal in U.S. Pat. No. 4,676,980.

### Methods of Treatment

The compounds described herein can be used to treat a subject. In certain embodiments the compounds described herein modulate the expression and/or activity of LRR superfamily molecules and, therefore, are useful for the treatment and/or prevention of infection, inflammation, septic shock, for modulating the expression of proinflammatory cytokines and for increasing the rate of wound healing.

Exemplary inflammatory conditions include, for example, multiple sclerosis, rheumatoid arthritis, psoriatic arthritis, degenerative joint disease, spondouloarthropathies, gouty arthritis, systemic lupus erythematosus, juvenile arthritis, rheumatoid arthritis, osteoarthritis, osteoporosis, diabetes (e.g., insulin dependent diabetes mellitus or juvenile onset diabetes), menstrual cramps, cystic fibrosis, inflammatory bowel disease, irritable bowel syndrome, Crohn's disease, mucous colitis, ulcerative colitis, gastritis, esophagitis, pancreatitis, peritonitis, Alzheimer's disease, shock, ankylosing spondylitis, gastritis, conjunctivitis, pancreatis (acute or chronic), multiple organ injury syndrome (e.g., secondary to septicemia or trauma), myocardial infarction, atherosclerosis, stroke, reperfusion injury (e.g., due to cardiopulmonary bypass or kidney dialysis), acute glomerulonephritis, vasculitis, thermal injury (i.e., sunburn), necrotizing enterocolitis, granulocyte transfusion associated syndrome, and/or Sjogren's syndrome. Exemplary inflammatory conditions of the skin include, for example, eczema, atopic dermatitis, contact dermatitis, urticaria, schleroderma, psoriasis, and dermatosis with acute inflammatory components.

As used herein, “wounds” include ulcers, bed sores, abscesses, burns, cuts, and surgical incisions. In specific embodiments, wounds include local septic wounds, e.g., septic ulcers or skin ulcers.

As used herein, “proinflammatory cytokines” include cytokines produced predominantly by activated immune cells such as microglia that are involved in the amplification of inflammatory reactions, e.g., IL-1, IL-6, TNF-α, and TGF-β.

As used herein, “bacterial infection” includes the detrimental, unwanted or undesirable colonization of tissue in a subject by bacteria. Exemplary bacterial infections are caused by staphylococcus or streptococcus.

As used herein, “inhibiting the expression or activity” of LRR superfamily proteins or nucleic acid molecules refers to a reduction, blockade of the expression or activity and does not necessarily indicate a total elimination of the LRR superfamily proteins or nucleic acid molecules expression or activity.

As used herein, “increasing the expression or activity” of LRR superfamily proteins or nucleic acids refers to the increase of expression or activity of a LRR superfamily protein or nucleic acid molecule, e.g., lumican.

The present invention provides for both prophylactic and therapeutic methods of treating a subject at risk of (or susceptible to) a disorder or having identified herein. As used herein, the term “treatment” is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease. A therapeutic agent includes, but is not limited to, full length LRR polypeptides, fragments of full length LRR polypeptides, nucleic acid molecules encoding LRR superfamily glycoproteins or fragments thereof, small molecules, peptides, antibodies, ribozymes, siRNA, shRNA and antisense oligonucleotides.

With regards to both prophylactic and therapeutic methods of treatment, such treatments may be specifically tailored or modified, based on knowledge obtained from the field of pharmacogenomics.

### EXAMPLES

It should be appreciated that the invention should not be construed to be limited to the examples that are now described; rather, the invention should be construed to include any and all applications provided herein and all equivalent variations within the skill of the ordinary artisan.

### Example 1

### Lumican Promotes the Recognition of Bacterial LPS and Host Innate Immune Response

Materials and Methods

The following were purchased from Sigma: Escherchia coli and Salmonella typhimurium lipopolysaccharides (LPS), Staphylococcus aureus peptidoglycan (PGN), N-acetylmuramyl-L-alanyl-D-isoglutamine hydrate (MDP), and Polyinosinic-polycytidylic acid sodium salt (Poly I:C). The phosphorothioate CpG-DNA (TCCATGACGTTCCTGATGCT) (SEQ ID NO:3) was obtained from Operon, recombinant mouse Tumor Necrosis Factor α (TNF-α) from Biosource, and recombinant Lumican (rLum) was prepared using the pSecTag2 vector in HEK 293 cells²².

Mouse Husbandry and Experimental Treatment

Lumican-deficient mice (Lum^{tm1/Chak} or Lum^{-/-}) were generated earlier by targeted gene disruption in 129Sv/J embryonic stem cell line¹¹, and subsequently crossed into the CD1 out bred mouse strain. All experiments investigating the effects on the null mutation were performed with Lum^{+/+} and Lum^{-/-} littermates generated by intercrossing heterozygous animals, unless stated otherwise. All animals were housed in the Johns Hopkins University specific pathogen-free mouse facility under conditions that were approved by the Association for Assessment and Accreditation of Laboratory Animal Care, and all animal procedures were approved by the Institutional Animal Care and use Committee.

To administer the LPS stimulant, LPS-saline solution at a final dose of 16.7 μg/g total body weight, and saline alone as control, were injected intraperitoneally. The mice were weighed daily for up to five days. For the cytokine analyses, the serum was harvested 32 hours after treatment and stored at 20° C. until use.

To test mice in a live bacteria challenge, S. typhimurium grown in tryptic soy broth and re-suspended in phosphate buffered saline, was injected intraperitoneally in a final volume of 200 μl to deliver 10⁶ bacteria per animal. The mice were euthanized on the fifth day, the spleen and liver were removed and homogenized; serial dilutions were plated on tryptic soy agar plates to obtain estimates of colony forming units (CFU) for each tissue.

Mouse Embryonic Fibroblasts

Primary cultures of mouse embryonic fibroblasts (MEF) were derived from E14 embryos²⁰. The MEFs were allowed to attach for 6 hours in DMEM F/12+10% FBS and then transferred to DMEM F/12+1% FBS. Cells between passages 2-6 were used.

Primary Macrophage Culture

Peritoneal macrophages were harvested from six to eight week old Lum^{+/+} and Lum^{-/-} littermates. A 4% thyioglycollate solution was injected into the peritoneal cavity (1 ml/mouse) and the peritoneal lavage harvested four days later. Cells were plated in 24-well tissue culture plates in RPMI 1640 medium and 1% FBS, at an initial density of 1.5×10⁵ cells/well. Twenty-four hours later non-adherent cells were removed and adherent cells were treated with specific PAMPs.

To obtain bone marrow-derived macrophages, 8 week old donor mice were sacrificed and bone marrow cells were harvested from both femurs. The cells were plated in RPMI1640 with 10% FBS and 10% L929 conditioned media. Non-adherent cells were harvested after 24 hours, plated in fresh RPMI 1640, cultured for an additional 3 days before assessment of NF-κB activation in response to LPS treatment.

Cytokine Measurements

Selected cytokines were measured by standard sandwich ELISA (enzyme linked-immuno-sorbent assay) in the serum or cell culture medium. Solid phase sandwich mouse ELISA kits for TNFα and IL-6, with a 3 pg/ml sensitivity were obtained from BioSource International, Inc. Total protein concentration was measured with the Bradford assay kit (BIO-RAD Laboratories, Inc.). The Beadlyte® Mouse Multi-Cytokine Flex Kit (Upstate Biotechnology) was used for multiplex cytokine profiling of IL-1β, IL-2, IL-4, IL-6, IL-10, IL-12, TNFα, IFN γ and GM-CSF from the serum. We acquired 100 assay points per cytokine for each sample using the Luminex® 100 IS System.

Quantitative RT-PCR

Total RNA was extracted using TRIzol Reagent (Invitrogen) from peritoneal macrophages and MEFs. The expression of lumican and glyceraldehydes 3 phosphate dehydrogenase (Gapdh), an internal reference gene, was determined by quantitative reverse-transcript polymerase chain reaction (RT-PCR) using iQ™SYBR Green Supermix Kit from BIO-RAD. The threshold cycle difference ΔCτ_{Lum}−Ct_{Gapdh}, and the relative expression was determined using the following formula: 2^{-ΔCtLum-ΔCτGapdh}. The following primers were used: Lum—forward 5′TCGAGCTTGATCTCTCCTAT3′ (SEQ ID NO:4) and reverse 5′ TGGTCCCAGGATCTTACAGAA3′ (SEQ ID NO:5) and Gapdh—forward 5′-TTGTCTCCTGCGACTTCA3′ (SEQ ID NO:6) and reverse 5′-CCTGITGCTGTAGCCGTATT3′ (SEQ ID NO:7).

Immunohistology

Mouse tissues were fixed in 10% buffered formalin for 4-6 hours, paraffin embedded and sectioned (6 μm thick) for conventional hematoxylin and eosin staining. For immunohistology of macrophage cultures, peritoneal macrophages were plated on glass coverslips, fixed in 4% paraformaldehyde and immunostained with 2 μg/ml of a rat monoclonal F/480 (Abeam, Inc., Cambridge, UK) or a rabbit polyclonal lumican antibody¹¹, followed by a secondary goat anti-rat Alexa Fluor 568 (green), or an anti-rabbit texas red, 5 μg/ml (Molecular Probes, Eugene, Oreg.) antibody, respectively. Negative controls consisted of identical treatments with the omission of the primary antibody. Hoechst dye, 1 μg/mL (Molecular Probes) was used for nuclear staining. The slides were then mounted (Vectashield; Vector Laboratories Inc.), and images were captured with quad filter settings as described before²².

Binding of rLum to Lum^{-/-} Macrophages

Lum^{-/-} peritoneal macrophages were harvested (1×10⁷ cells/ml) and incubated with rLum (20 μg/ml) or BSA as a control at room temperature for 1 hour. The cells were washed three times in PBS and incubated with 1 mM 3,3′ dithiobis sulfosuccinimidyl propionate as a cross-linker at room temperature for 2 30 min. The cells were washed, lysed and presence of rLum in the cell extract quantified by ELISA. 96-well microplates were coated with a goat polyclonal anti-lumican antibody (Santa Cruz Biotechnologies). Macrophage extracts of rLum treated and appropriate control cells were added to the wells, washed and the presence of rLum detected using a rabbit anti-lumican against a human lumican-derived synthetic peptide that recognizes rLum. Biotinylated goat anti-rabbit IgG (R &D Systems) was used as the secondary antibody to determine amount of rLum retained in the wells.

Electrophoresis Mobility Gel Shift Assay (EMSA)

Bone marrow-derived macrophages (1×10⁵ cells/ml in 60 mm2 tissue culture dishes) were exposed to 10 ng/ml of E. coli LPS in culture. For the electrophoretic mobility gel shift assays (EMSA) nuclear extracts of the macrophages were incubated with γ³²P end-labeled NF-κB binding-consensus sequence oligonucleotide and a mutant non-binding control oligonucleotide (SC-2505, Sc2511, Santa Cruz Biotechnology Inc.). DNA protein complexes were resolved by 4% polyacrylamide gel electrophoresis.

LPS-Binding Assay

Recombinant lumican (1 μg/ml in 0.1 M NaHCO₃ and 2.5 mM Na₂CO₃, pH9.6, 100 ul/well) was used to coat Corning Costar 96-well plates (polystyrene, with black walls, Fisher Scientific Co.,) overnight at 4° C. After three washes with PBS containing 0.2% Tween, lumican-coated and uncoated wells were blocked with 3% BSA in PBS for 2 h at room temperature. FITC-labeled E. Coli LPS (Sigma-Aldrich Co.), at 0.0625-1 μg/ml in Hanks' balance salt solution (100 μl/well) was added and incubated for 1 h at room temperature followed by three washes. Fluorescence was measured by a SpectraMax M2 microplate reader (Molecular Devices Co., Sunnyvale, Calif., USA) with 485 nm for excitation and 525 nm for emission wavelengths. Experiments were replicated twice and results are shown as relative fluorescence units normalized to a set of reference wells.

Statistical Methods

Numbers of mice and the data for each experiment are provided in figure legends. To compare the difference between two groups, we used the Student's t-test with the assumptions of unequal variances. A p value <0.05 was considered statistically significant.

Lum^{-/-} Binding Mice are Hypo-Responsive to Bacterial LPS

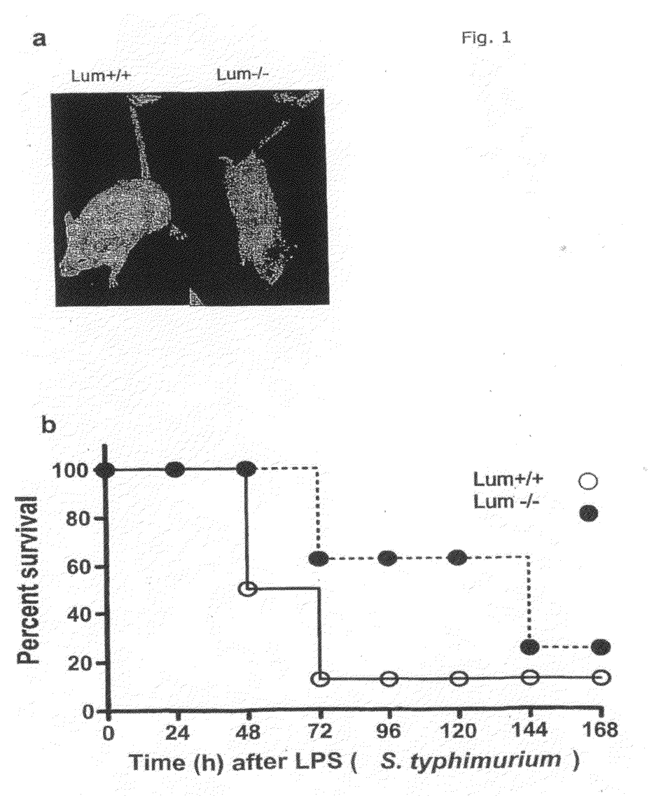

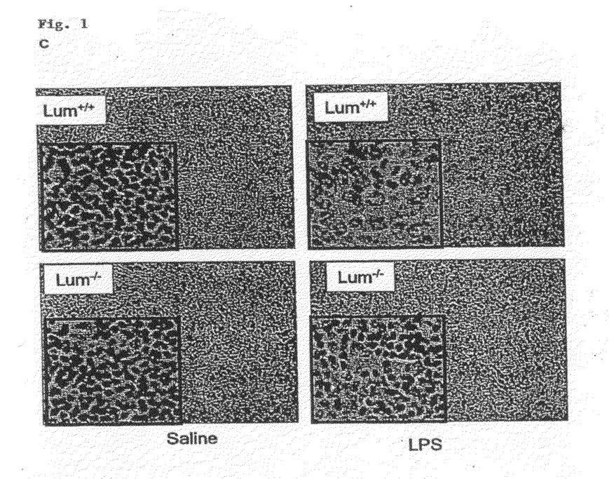

We tested Lum^{-/-} mice in a septic shock model to investigate the involvement of lumican in innate immune functions. Seven-week old Lum^{-/-} and Lum^{+/+} littermate mice were given a single intraperitoneal injection of S. typhimurium LPS. Within 24 to 36 hours the Lum^{+/+} mice were showing piloerection and other visible signs of sickness, while the Lum^{-/-} mice appeared healthy with little sign of distress (FIG. 1a). In the Lum^{+/+} mice most deaths occurred within 48 to 72 hours of exposure to the endotoxin (FIG. 1b). A small number of the Lum^{-/-} mice died by 72 hours, but overall a higher percentage of the mutants survived. Exposure to E. Coli LPS had similar mild effects on the Lum^{-/-} mice, but caused septic shock and death in the Lum^{+/+} mice (not shown).

Histology of the spleen from Lum^{+/+} mice challenged with LPS revealed florid follicular hyperplasia with germinal centers, tingible body macrophages and a predominance of immunoblasts and plasmacytoid cells. In contrast, the sections of the spleen from Lum^{-/-} mice challenged with LPS contained minimally reactive follicles, and lacked germinal centers and other histologic features typically associated with an immune response (FIG. 1c).

Impaired Induction of Pro-Inflammatory Serum Cytokines in LPS-Treated Lum^{-/-} Mice

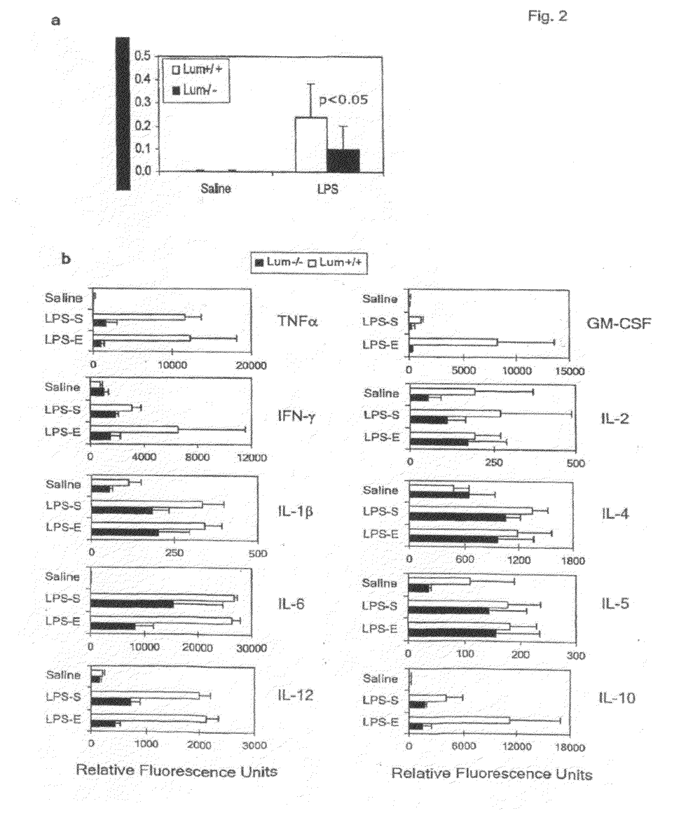

To assess innate immune response of mice challenged with LPS, TNFα was measured in the serum of Lum^{-/-} and Lum^{+/+} littermate animals, 32 hours after an intraperitoneal injection of E. coli LPS. The Lum^{-/-} lumican-deficient mice presented significantly (p=0.028) lower levels of TNFα in the serum (FIG. 2a). A multiplex cytokine analysis of mice (age and gender matched Lum^{+/+} and Lum^{-/-} non-littermates) challenged with S. typhimurium or E. coli LPS further revealed poor induction of IFN γ, IL-1β, IL-12, IL-6, IL-10 and GM-CSF in the Lum^{-/-} mice (FIG. 2b). Thus, a broad repertoire of proinflammatory cytokines, and those associated with T_{H}2 T cell functions are induced in wild type Lum^{+/+} mice but not induced optimally in the Lum^{-/-} mice in response to a systemic challenge of LPS. The impaired induction of these cytokines is the likely cause for reduced LPS-septic shock in the Lum^{-/-} mice.

Poor Induction of TNFα and IL-6 in Lum^{-/-} Macrophages Treated with LPS

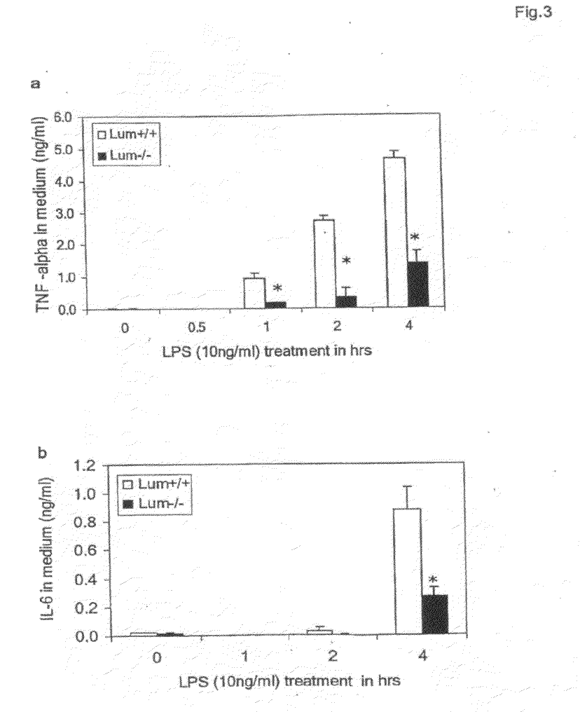

Primary cultures of elicited peritoneal macrophages from Lum^{-/-} and Lum^{+/+} littermates were exposed to E. coli LPS, and the release of pro-inflammatory cytokines, TNFα and IL-6 into the medium was measured by ELISA at different time points. The temporal induction of both TNFα and IL-6, in response to 10 ng/ml of LPS, was significantly lower in the Lum^{-/-} macrophage cultures (FIG. 3a, b).

Recombinant Lumican Rescues LPS-Mediated TNFα Induction in Lum^{-/-} Macrophages

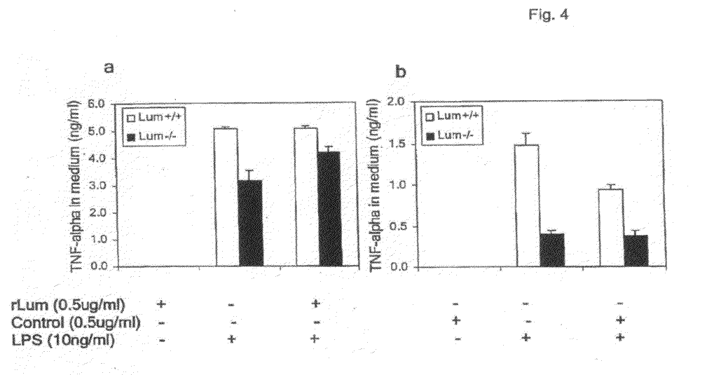

To test if exogenous lumican is able to restore LPS sensitivity to the Lum^{-/-} macrophages, we measured TNFα induction in the presence of recombinant lumican (rLum). Addition of rLum, to the culture medium, at the time of LPS treatment, significantly increased TNFα in the LPS treated Lum^{-/-} macrophages (FIG. 4). The rLum protein alone did not induce TNFα in the Lum^{+/+} or Lum^{-/-} macrophages. Ovalbumin, used as a non-lumican control protein, had no such “rescue effect” on TNFα induction in the Lum^{-/-} macrophages (FIG. 4).

Lum^{-/-} Macrophages Respond Normally to Other PAMPs

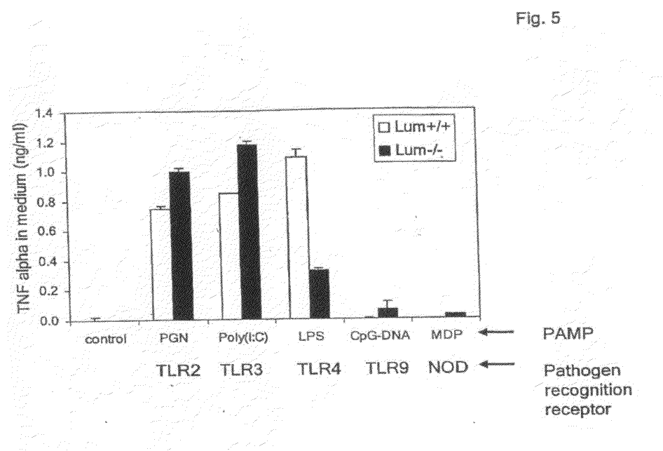

To determine whether there is some specificity in the TLR pathways affected by lumican, we challenged Lum^{+/+} and Lum^{-/-} peritoneal macrophages with a panel of PAMPs, recognized by different cell-surface TLR molecules (FIG. 5). With the exception of LPS, the induction of TNFα in response to all other PAMPs was comparable in the Lum^{+/+} and Lum^{-/-} macrophages. Therefore, it appears that only TLR4-mediated recognition of LPS is compromised by lumican-deficiency.

Diminished Activation of NF-κB in Lum^{-/-} Macrophages

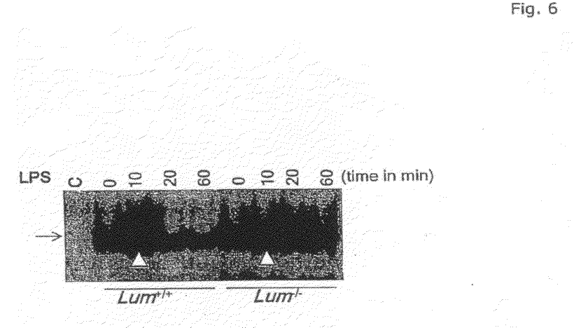

The activation of the transcription factor NF-κB and its nuclear localization is a major route to the transcriptional up regulation of cytokine genes and induction of pro-inflammatory cytokines in response to LPS. Therefore, we assayed for NF-κB-DNA binding activity in Lum^{+/+} and Lum^{-/-} bone marrow-derived macrophages at different time points after LPS stimulation by electrophoretic mobility gel shift assays (FIG. 6). The nuclear localization and DNA binding activity of NF-κB was delayed in Lum^{-/-} macrophages.

Response of Lum^{-/-} Mice to Live Bacteria

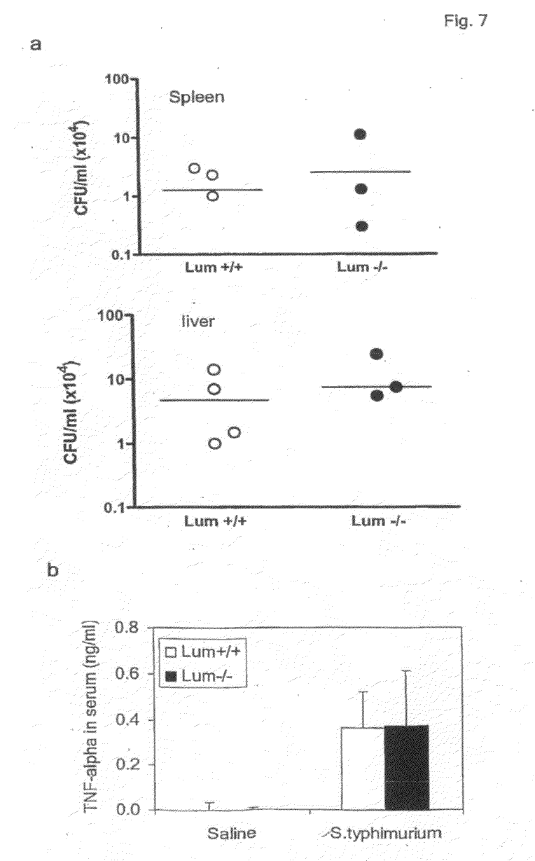

We tested the response of Lum^{-/-} mice to live bacteria by administering a single intraperitoneal injection of S. typhimurium. Compared to Lum^{+/+} mice, there was a small increase in bacterial yield from the liver and spleen of infected Lum^{-/-} mice (FIG. 7a). However, measurements of TNFα indicated no difference in its induction in Lum^{-/-} versus Lum^{+/+} mice (FIG. 7b). Therefore, although LPS-recognition and stimulation of innate immunity is impaired in the Lum^{-/-} mice, response to whole bacteria appear to occur relatively unhindered. These findings are similar to previously published studies on the CD14-null mice that revealed a divergent response to monomeric LPS and whole bacterial particles. There was a failure to recognize LPS and to induce TNFα in the CD14-nulls, on the one hand, but unmitigated response to whole bacteria and induction of TNFα on the other hand²⁹. It appears that CD14 regulates the efficient recognition of monomeric LPS, leading to the induction of an innate immune response. However, whole bacteria or large aggregates can induce the same down stream events to innate immune response by multiple mechanisms: signaling via other receptors such as the CD11/CD18 integrins, or the direct phagocytic internalization of pathogenic stimulants^{26,29}. Similarly, in the Lum^{-/-} mice, while LPS response is compromised, the animals are able to counteract bacterial infection through alternative innate immune response mechanisms.

Lumican is an Innate Immune Response Protein

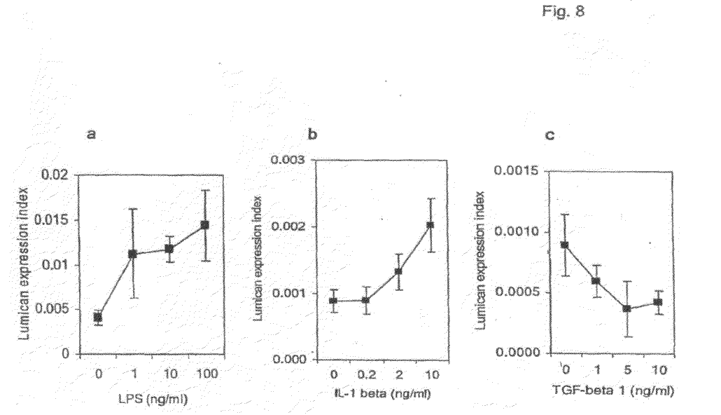

Lumican is a ubiquitous ECM protein expressed by fibroblasts. Since lumican-deficiency has a profound effect on innate immune response to LPS, we tested if lumican expression is inducible by LPS and the pro-inflammatory cytokine IL-1β. In mouse embryonic fibroblasts lumican expression was elevated after LPS (FIG. 8a) and IL-1β treatment (FIG. 8b), but inhibited by TGFβ (FIG. 8c). Thus lumican may be a member of the arsenal of host proteins that are induced during an innate immune response.

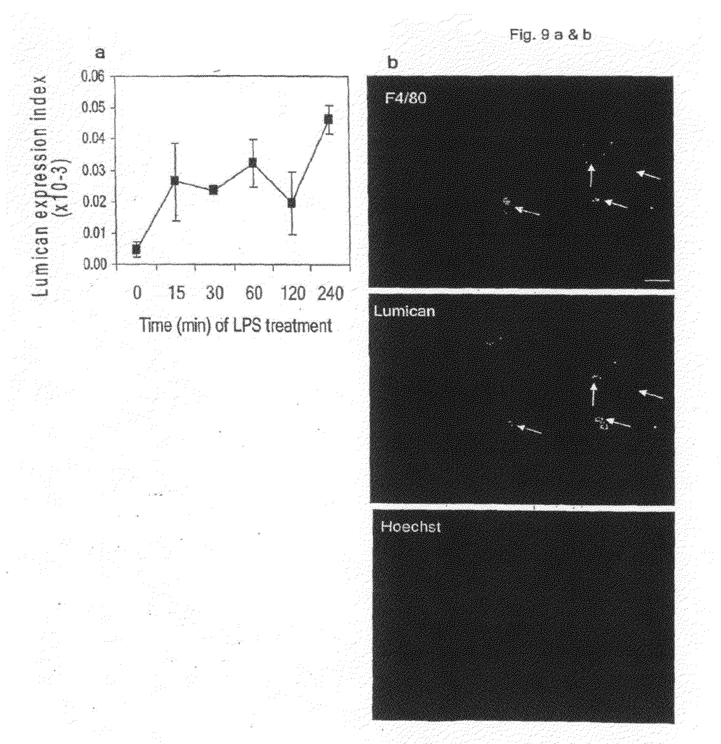

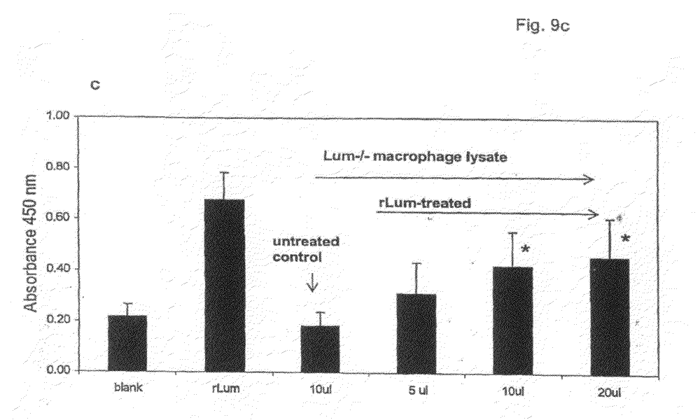

Macrophages are key mediators of innate immune response. Our results indicated a compromise in innate immune response to LPS in Lum^{-/-} macrophages. However, macrophages, even after LPS stimulation express little lumican as shown by qRT-PCR measurements of lumican mRNA in Lum^{+/+} macrophages (FIG. 9a). To modulate the LPS-TLR4 signaling pathway lumican is likely to be associated with macrophage cell surfaces. Immunostaining of peritoneal macrophages with an anti-lumican antibody showed that a subset of F4/80 positive (macrophage marker) cells were positive for lumican (FIG. 9b), indicating the presence of lumican on macrophages. We further tested the ability of exogenous recombinant lumican to associate with Lum^{-/-} macrophage in culture (FIG. 9b). Lum^{-/-} macrophage cultures were incubated with rLum, treated with a cross-linker to stabilize protein-protein interactions at the cell surface, and the cell extracts were tested for the presence of macrophage-bound rLum by ELISA. Lum^{-/-} macrophages not treated with rLum showed no rLum reactivity as expected (untreated control, FIG. 9c). Extracts of Lum^{-/-} macrophage cultures incubated with rLum showed rLum reactivity that increased with the total amount of extract used in the ELISA, indicating a specific association between recombinant lumican and Lum^{-/-} macrophages. A previous study has also shown binding of macrophages to lumican³⁰.

Lumican Binds LPS

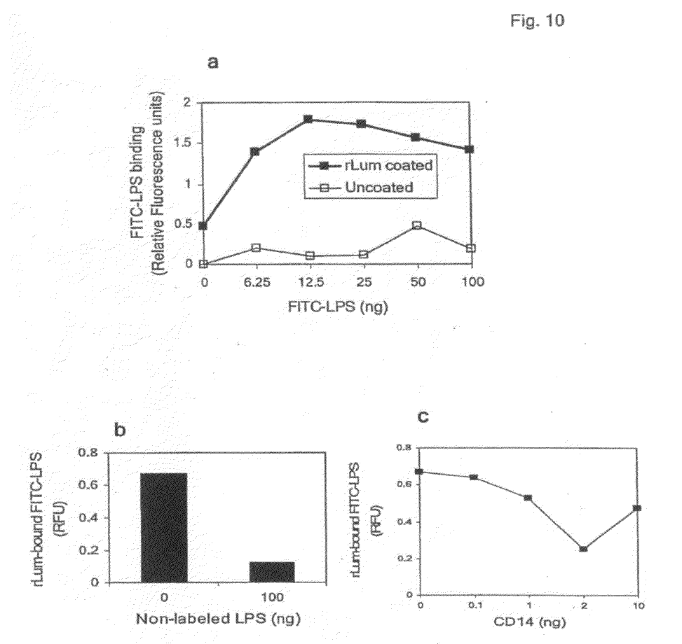

To test our hypothesis that lumican binds LPS and facilitates its presentation to the LPS signaling complex at the cell surface, we tested the binding of lumican with LPS in a direct solid-phase binding assay. Increasing concentrations of FITC-LPS was incubated in 96-well plates coated with lumican. The wells were washed, and bound LPS retained in the wells, detected by measuring the fluorescence. There was little retention of FITC-LPS in the control uncoated wells blocked with BSA, while lumican-coated wells bound and retained FITC-LPS (FIG. 10a). Furthermore binding of FITC-LPS to rLum could be specifically competed out by excess of unlabeled LPS (FIG. 10b). When soluble CD14 was included in the lumican-LPS binding assays, there was a CD14-dose dependent decrease in the retention of FITC-LPS in the rLum-coated wells, indicating a replacement of rLum-LPS interactions by competitive binding of CD14 to FITC-LPS (FIG. 10c).

### Summary

The experiments described herein have identified a novel role for an ECM protein, lumican, in promoting recognition of bacterial LPS and host innate immune response. The results demonstrate that Lum^{-/-} mice are impaired in inducing proinflammatory cytokines and are hyporesponsive to LPS. Biglycan, another ECM protein was also implicated recently in aiding response to LPS³¹. Lumican and biglycan represent a large group of ECM proteins and proteoglycans that belong to the LRR superfamily. Notable LRR proteins known to regulate innate immune response are the TLR receptors and CD14 at the cell surface, and the Nod proteins in the cytoplasm. The biglycan and our lumican study have now identified LRR protein regulators of innate immune response in the ECM not recognized before.

These experiments demonstrate that host innate immune response is regulated by lumican, an ECM protein of the LRR superfamily.

### Example 2

### Identifying Functional Fragments of Lumican

The data presented herein demonstrates that lumican interacts at the cell surface with CD14 and that this interaction is important to LPS signaling. The following observations support. 1) The core protein of lumican binds Mφ specifically. The rLum (Vij, 2004, Appendix) we made is a glycoprotein and not a proteoglycan, and it still binds LPS and is biologically active. 2) Immunostain shows lumican on Mφ surface Preliminary studies). 3) Immunoprecipitation of lumican from extracts of resting Mφ co-precipitates CD14. The surprise was that after 2 hours of LPS treatment, CD14 could no longer be detected in the lumican-IP complex, suggesting that lumican-CD14 interactions are dynamic and that cell surface changes upon macrophage activation by LPS, lead to a loss of this interaction. 4) rLum binds LPS in solid phase binding assays, and when rCD14 is added it can successfully compete for LPS from lumican. However, while evidence supports binding of lumican to LPS and also lumican-CD14 interactions on resting Mφ and that lumican-deficiency leads to a functional impairment in innate immune response to LPS, the mechanistic details of how lumican aids in the transduction of the LPS signal and the biological implications of its interactions with CD14 are not clear.

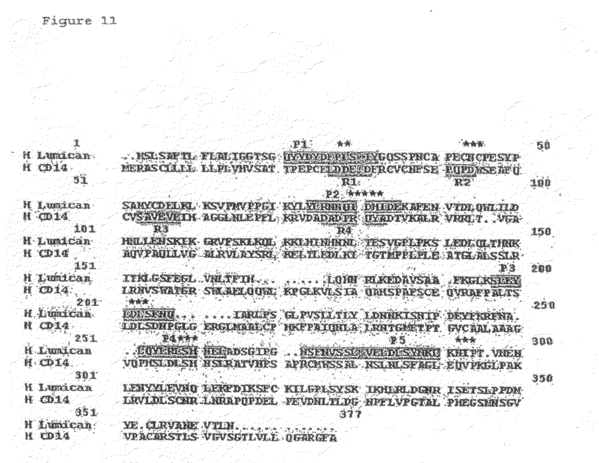

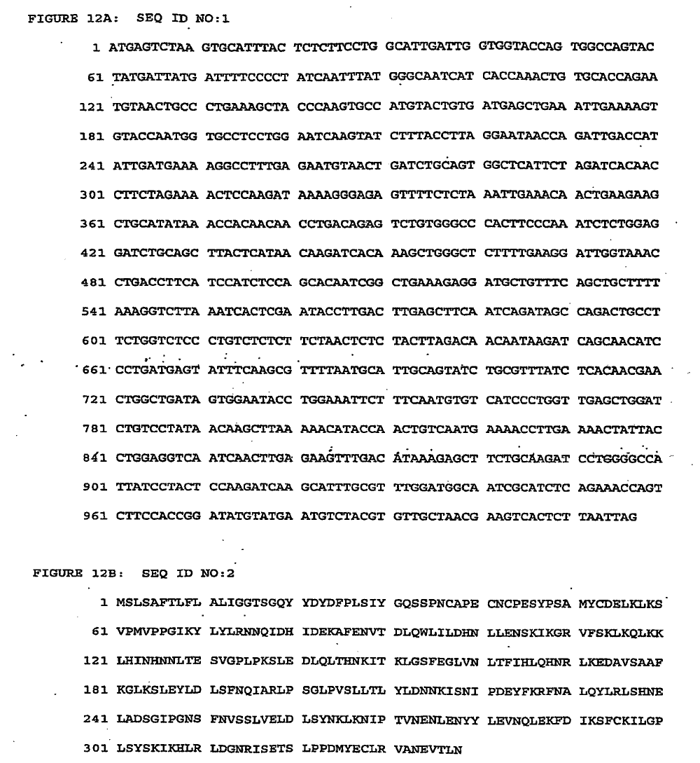

To determine the temporal dynamics of lumican-CD14 interactions (by co-immunoprecipitation), and the CD14 and LPS binding sites on lumican using rLum, mutated rLum, synthetic peptides in solid-phase binding assays (FIG. 12).

Experimental design. rLum variants and synthetic peptides will be used for this aim. rLum was made by expressing a human LUM cDNA clone. This human recombinant lumican protein is biologically active on mouse Mφ. Similarly, many other studies have shown functional cross-reactivity of highly conserved biological functions of proteins, as in Fas, for example. In order to identify functional fragments and variants, the following experiments will be preformed.

Point mutations or 2-3 aa deletions (asterisk, FIG. 11), within the 30-60aa region at the N-terminus, which shows sequence similarity with the LPS-binding site of CD14, will be made. Further mutations will be made in the 200-300aa segment. This area in CD14 binds proteins of the LPS-signal transduction mechanism. Lumican shows three areas of sequence identity with CD14 in this 200-300aa region that may be involved in CD14 binding.

Elucidate lumican-CD14 interaction dynamics: Peritoneal Mφ lavagerich in lumican) or a Mφ cell line (J774, ATCC) in the presence of rLum will be treated with LPS for varying times (0 to 2 h), total protein extracted and immunoprecipitated (IP) for lumican and check for CD14 by immunoblotting. This will indicate when after LPS, the lumican CD14 interactions are lost. We will also IP CD14 and then blot for lumican. The direct CD14 IP will indicate If LPS stimulation leads to shedding of CD14 and that is the reason for loss of cell surface associated lumican. In a second approach the J774 cell line will be co-transfected with Lum and CD14 construct and then IP lumican and immunoblot for CD14 or IP CD14 and test for pull down of lumican. This cell line itself does not make any lumican. Expression of lumican and CD14 will be monitored after transfection and then used for the co-IP experiments.

CD14 binding sites on lumican: a) The J 774 Mφ line will be transfected with the Lum construct or those coding for mutated forms of lumican, and co-transfected with the CD14 construct. The co-transfected cell line will be tested for expression of the mutated lumican forms first, and then induced with LPS, followed by lumican or CD14 IP and immunoblotting to test for binding between the mutated lumican forms and CD14. b) Synthetic peptides will also be used to test whether these mimic the CD14 binding sites on lumican.

c) The effects of adding these to the culture medium of the co-transfected J774 cells to see if these interfere with lumican-CD14 interactions in a specific way will be monitored. d) A lumican antibody will be generated targeting the 200-300 aa region that shows increased similarity with CD14. This antibody will be tested to see if it can block lumican-CD14 interactions and inhibit LPS-mediated TNFα induction by Mφ cultures.

Lumican-LPS binding: We will use the 10-12 amino acid-long synthetic peptides (FIG. 12) and their corresponding scrambled-sequence control peptides as controls in sold phase binding assays to determine if they either bind LPS or CD14. Or, use the synthetic peptides in competition binding assays where binding of FITC-LPS to the full length rLum will be assessed in the presence of varying doses of the peptides.

### REFERENCES

1. Janeway, C. A., Jr. & Medzhitov, R. Innate immune recognition. Annu Rev Immunol 20, 197-216 (2002).
2. Medzhitov, R. Toll-like receptors and innate immunity. Nat Rev Immunol 1, 135-145 (2001).
3. Inohara, N. & Nunez, G. Nods: Intracellular proteins involved in inflammation and apoptosis. Nat Rev Immunol 3, 371-382 (2002).
4. Werling, D. & Jungi, T. W. Toll-like receptors linking innate and adaptive immune response. Vet Immunol Immunopathol 91, 1-12 (2003).
5. Takeda, K. & Akira, S. Tlr signaling pathways. Semin Immunol 16, 3-9 (2004).
6. Nurnberger, T., Brunner, F., Kemmerling, B. & Piater, L. Innate immunity in plants and animals: Striking similarities and obvious differences. Immunol Rev 198, 249-266 (2004).
7. Inohara, Chamaillard, McDonald, C. & Nunez, G. Nod-lrr proteins: Role in host-microbial interactions and inflammatory disease. Annu Rev Biochem 74, 355-383 (2005).
8. Kobayashi, K. et al. Rick/rip2/cardiak mediates signalling for receptors of the innate and adaptive immune systems. Nature 416, 194-199 (2002).
9. Ogura, Y. et al. Nod2, a nod1/apaf-1 family member that is restricted to monocytes and activates nf-kappab. J Biol Chem 276, 4812-4818. (2001).
10. Iozzo, R. V. The biology of the small leucine-rich proteoglycans. Functional network of interactive proteins. J Biol Chem 274, 18843-18846. (1999).
11. Chakravarti, S. et al. Lumican regulates collagen fibril assembly: Skin fragility and corneal opacity in the absence of lumican. J Cell Biol 141, 1277-1286 (1998).
12. Blochberger, T., Cornuet, P. & Hassell, J. Isolation and partial characterization of lumican and decorin from adult chicken corneas. J Biol. Chem. 267, 20613-20619 (1992).
13. Funderburgh, J., Funderburgh, M., Mann, M. & Conrad, G. Arterial lumican. J Biol. Chem. 266, 24773-24777 (1991).
14. Ezura, Y. et al. Differential expression of lumican and fibromodulin regulate collagen fibrillogenesis in developing mouse tendons. J Cell Biol 151, 779-788. (2000).
15. Jepsen, K. J. et al. A syndrome of joint laxity and impaired tendon integrity in lumican- and fibromodulin-deficient mice. J Biol Chem 277, 35532-35540 (2002).
16. Rada, J. A., Cornuet, P. K. & Hassell, J. H. Regulation of corneal collagen fibrillogenesis in vitro by corneal keratan sulfate proteoglycan (lumican) and decorin core proteins. Exp. Eye Res. 56, 635-648 (1993).
17. Svensson, L., Narlid, I. & Oldberg, A. Fibromodulin and lumican bind to the same region on collagen type i fibrils. FEBS Lett 470, 178-182. (2000).
18. Chakravarti, S. et al. Ocular and scleral alterations in gene-targeted lumican-fibromodulin double-null mice. Invest Opthalmol Vis Sci 44, 2422-2432 (2003).
19. Chakravarti, S. et al. Corneal opacity in lumican-null mice: Defects in collagen fibril structure and packing in the posterior stroma. Invest Opthalmol Vis Sci 41, 3365-3373 (2000).
20. Vij, N., Roberts, L., Joyce, S. & Chakravarti, S. Lumican suppresses cell proliferation and aids fas-fas ligand mediated apoptosis: Implications in the cornea. Exp Eye Res 78, 957-971 (2004).
21. Santra, M., Eichstetter, I. & Iozzo, R. V. An anti-oncogenic role for decorin. Down-regulation of erbb2 leads to growth suppression and cytodifferentiation of mammary carcinoma cells. J Biol Chem 275, 35153-35161. (2000).
22. Vij, N., Roberts, L., Joyce, S. & Chakravarti, S. Lumican regulates corneal inflammatory responses by modulating fas-fas ligand signaling. Invest Opthalmol Vis Sci 46, 88-95 (2005).
23. Raetz, C. R. & Whitfield, C. Lipopolysaccharide endotoxins. Annu Rev. Biochem 71, 635-700 (2002).
24. Beutler, B., Du, X. & Poltorak, A. Identification of toll-like receptor 4 (tlr4) as the sole conduit for lps signal transduction: Genetic and evolutionary studies. J Endotoxin Res 7, 277-280 (2001).
25. Triantafilou, M. & Triantafilou, K. Lipopolysaccharide recognition: Cd14, tlrs and the lps-activation cluster. Trends Immunol 23, 301-304 (2002).
26. Triantafilou, K., Triantafilou, M. & Dedrick, R. L. A cd14-independent lps receptor cluster. Nat Immunol 2, 338-345 (2001).
27. Viriyakosol, S. & Kirkland, T. N. A region of human cd14 required for lipopolysaccharide binding. J Biol Chem 270, 361-368 (1995).
28. Triantafilou, M. & Triantafilou, K. Heat-shock protein 70 and heat-shock protein 90 associate with toll-like receptor 4 in response to bacterial lipopolysaccharide. Biochem Sac Trans 32, 636-639 (2004).
29. Moore, K. J. et al. Divergent response to lps and bacteria in cd14-deficient murine macrophages. J Immunol 165, 4272-4280 (2000).
30. Funderburgh, J. L. et al. Macrophage receptors for lumican. A corneal keratan sulfate proteoglycan. Invest Opthalmol Vis Sci 38, 1159-1167. (1997).
31. Schaefer, L. et al. The matrix component biglycan is proinflammatory and signals through toll-like receptors 4 and 2 in macrophages. J Clin Invest 115, 2223-2233 (2005).
32. Chakravarti, S. et al. Primary structure of human lumican (keratan sulfate proteoglycan) and localization of the gene (lum) to chromosome 12q21.3-q22. Genomics 27, 481-488. (1995).
33. Kim, J. I. et al. Crystal structure of cd14 and its implications for lipopolysaccharide signaling. J Biol Chem 280, 11347-11351 (2005).
34. Choe, J., Kelker, M. S. & Wilson, I. A. Crystal structure of human toll-like receptor 3 (tlr3) ectodomain. Science 309, 581-585 (2005).
35. Scott, P. G. et al. Crystal structure of the dimeric protein core of decorin, the archetypal small leucine-rich repeat proteoglycan. Proc Natl Acad Sci USA 101, 15633-15638 (2004).
36. Jiang, D. at al. Regulation of lung injury and repair by toll-like receptors and hyaluronan. Nat Med 11, 1173-1179 (2005).
37. O'Neill, L. A. Tim play good cop, bad cop in the lung. Nat Med 11, 1161-1162 (2005).
38. Fukata, M. et al. Toll-like receptor-4 is required for intestinal response to epithelial injury and limiting bacterial translocation in a murine model of acute colitis. Am J Physiol Gastrointest Liver Physiol 288, G1055-1065 (2005).

### INCORPORATION BY REFERENCE

The contents of all references, patents, pending patent applications and published patents, cited throughout this application are hereby expressly incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method of treating inflammation in a subject comprising:
administering to the subject an effective amount of a composition that decreases the activity or expression of a LLR superfamily glycoprotein, wherein the glycoprotein binds to LPS;
thereby treating the inflammation in the subject.

2. A method of modulating activity or expression of proinflammatory cytokines in a subject comprising:
administering to the subject an effective amount of a composition that decreases the activity or expression of a LRR superfamily glycoprotein, wherein the glycoprotein binds to LPS;
thereby modulating the activity or expression of proinflammatory cytokines in the subject.

3. A method of increasing the rate of wound healing comprising:
administering to the subject an effective amount of a composition that increases the activity or expression of a LRR superfamily glycoprotein, wherein the glycoprotein binds to LPS;
thereby increasing the rate of wound healing in a subject.

4. A method of treating a subject having a bacterial infection comprising:
administering to the subject an effective amount of a composition that decreases the activity or expression of a LRR superfamily glycoprotein, wherein the glycoprotein binds to LPS;
thereby treating the subject.

5. A method of treating a subject having septic shock comprising:
administering to the subject an effective amount of a composition that decreases the activity or expression of a LRR superfamily glycoprotein, wherein the glycoprotein binds to LPS;
thereby treating the subject.

6. The method of claim 1, wherein the composition is a LRR polypeptide or nucleic acid molecule or a fragment thereof, peptide mimetic, antibody, small molecule, antisense RNA, siRNA, shRNA, ribozyme or aptamer.

7. The method of claim 1, wherein the LRR polypeptide is lumican.

8. The method of claim 1, wherein the lumican has the polypeptide set forth as SEQ ID NO:2, or a fragment thereof.

9. The method of claim 8, wherein the lumican fragment comprises a LRR repeat comprising the sequence XL²XXL⁵XL⁷XXN¹⁰XL.

10-12. (canceled)

13. A pharmaceutical composition comprising (1) a lumican polypeptide or nucleic acid molecule, a fragment thereof, or a lumican modulator, or (2) an siRNA, antisense RNA₅ or shRNA specific for a lumican encoding nucleic acid.

14-16. (canceled)

17. A kit:
(1) for the treatment of inflammation comprising a lumican modulator and instructions for use; or
(2) for the treatment of wound healing comprising a comprising a lumican modulator and instructions for use; or
(3) for increasing the expression of proinflammatory cytokines comprising a lumican modulator and instructions for use; or
(4) for the treatment of a bacterial infection comprising a comprising a lumican modulator and instructions for use; or
(5) kit for the treatment of septic shock comprising a lumican modulator and instructions for use.

18-21. (canceled)

22. The kit of claim 17, wherein the composition is a LRR superfamily glycoprotein or nucleic acid fragment, or fragment thereof, peptide mimetic, antibody, small molecule, antisense RNA, siRNA, shRNA, ribozyme or aptamer.

23. The kit of claim 22, wherein the LRR superfamily glycoprotein is lumican.

24. The kit of claim 23, wherein the lumican has the polypeptide set forth as SEQ ID NO:2, or a fragment thereof.

25. The kit of claim 24, wherein the lumican fragment comprises a LRR repeat comprising the sequence XL²XXL⁵XL⁷XXN¹⁰XL.

26. The kit of claim 22, wherein the antibody is a blocking antibody.

27. The kit of claim 22, wherein the antibody is a monoclonal antibody, polyclonal antibody, or fragment thereof.

28-30. (canceled)
